(19) United States
(12) Patent Application Publication
(10) Pub. No.: US 2024/0128521 A1
(43) Pub. Date: Apr. 18, 2024

(54) COMPOSITION

(71) Applicant: Mexichem Fluor S.A. de C.V., San Luis Potosi (MX)

(72) Inventors: Andrew SHARRATT, Runcorn (GB); Miodrag OLJACA, Concord, MA (US); Ira SAXENA, Runcorn (GB)

(21) Appl. No.: 17/768,784

(22) PCT Filed: Oct. 15, 2020
(86) PCT No.: PCT/GB2020/052595

§ 371 Date: Apr. 13, 2022

Related U.S. Application Data
(60) Provisional application No. 62/916,459, filed on Oct. 17, 2019.

(30) Foreign Application Priority Data
Nov. 7, 2019 (GB) 1916221.3
May 1, 2020 (GB) 2006445.7

(51) Int. Cl. H01M 10/42; H01M 10/0569; H01M 50/383

(52) U.S. Cl. CPC H01M 10/4235; H01M 10/0569; H01M 50/383

(57) ABSTRACT

Use of a compound of Formula 1 in a nonaqueous battery electrolyte formulation (1) wherein each R¹ to R⁴ is selected from the group consisting of F, Cl, H, CF₃, and C₁ to C₆ alkyl which may be at least partially fluorinated, wherein at least one of R¹ to R⁴ is or comprises F.

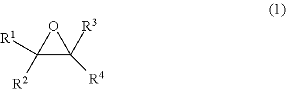

The present disclosure relates to nonaqueous electrolytic solutions for energy storage devices, including batteries and capacitors, especially for secondary batteries and devices known as supercapacitors.

There are two main types of batteries; primary and secondary. Primary batteries are also known as non-rechargeable batteries. Secondary batteries are also known as rechargeable batteries. A well-known type of rechargeable battery is the lithium-ion battery. Lithium-ion batteries have a high energy density, no memory effect and low self-discharge.

Lithium-ion batteries are commonly used for portable electronics and electric vehicles. In the batteries lithium ions move from the negative electrode to the positive electrode during discharge and back when charging.

Typically, the electrolytic solutions include a nonaqueous solvent and an electrolyte salt plus additives. The electrolyte is typically a mixture of organic carbonates such as ethylene carbonate, propylene carbonate, fluoroethylene carbonate and dialkyl carbonates containing a lithium ion electrolyte salt. Many lithium salts can be used as the electrolyte salt and common examples include lithium hexafluorophosphate (LiPF₆), lithium bis (fluorosulfonyl) imide “LiFSI” and lithium bis(trifluoromethanesulfonyl)imide (LiTFSI).

The electrolytic solution has to perform a number of separate roles within the battery.

The principal role of the electrolyte is to facilitate the flow of electrical charge between the cathode and anode. This occurs by transportation of metal ions within the battery from and or to one or both of the anode and cathode, where by chemical reduction or oxidation, electrical charge is liberated/adopted.

Thus the electrolyte needs to provide a medium which is capable of solvating and/or supporting the metal ions.

Due to the use of lithium electrolyte salts and the interchange of lithium ions with lithium metal; which is very reactive with water, as well as the sensitivity of other battery components to water; the electrolyte is usually non-aqueous.

Additionally the electrolyte has to have suitable rheological properties to permit/enhance the flow of ions therein; at the typical operating temperature to which a battery is exposed and expected to perform.

Moreover the electrolyte has to be as chemically inert as possible. This is particularly relevant, in the context of the expected lifetime of the battery, in regard to internal corrosion within the battery (e.g. of the electrodes and casing) and the issue of battery leakage. Also of importance within the consideration of chemical stability is flammability. Unfortunately typical electrolyte solvents can be a safety hazard since they often comprise a flammable material.

This can be problematic as in operation when discharging or being discharged, batteries may accumulate heat. This is especially true for high density batteries such as lithium ion batteries. It is therefore desirable that the electrolyte displays a low flammability, with other related properties such as a high flash point.

It is also desirable that the electrolyte does not present an environmental issue with regard to disposability after use or other environmental issue such as global warming potential.

It is an object of the present invention to provide a nonaqueous electrolytic solution, which provides improved properties over the nonaqueous electrolytic solution of the prior art.

### USE ASPECTS

According to a first aspect of the invention there is provided the use of a compound of Formula 1 in a nonaqueous battery electrolyte formulation.

According to a second aspect of the invention there is provided the use of a nonaqueous battery electrolyte formulation comprising a compound of Formula 1 in a battery.

### COMPOSITION/DEVICE ASPECTS

According to a third aspect of the invention there is provided a battery electrolyte formulation comprising a compound of Formula 1.

According to a fourth aspect of the invention there is provided a formulation comprising a metal ion and a compound of Formula 1, optionally in combination with a solvent.

According to a fifth aspect of the invention there is provided a battery comprising a battery electrolyte formulation comprising a compound of Formula 1.

### METHOD ASPECTS

According to a sixth aspect of the invention there is provided a method of reducing the flash point of a battery and/or a battery electrolyte formulation, comprising the addition of a formulation comprising a compound of Formula 1.

According to a seventh aspect of the invention there is provided a method of powering an article comprising the use of a battery comprising a battery electrolyte formulation comprising a compound of Formula 1.

According to an eighth aspect of the invention there is provided a method of retrofitting a battery electrolyte formulation comprising either (a) at least partial replacement of the battery electrolyte with a battery electrolyte formulation comprising a compound of Formula 1 and/or (b) supplementation of the battery electrolyte with a battery electrolyte formulation comprising a compound of Formula 1.

According to a ninth aspect of the invention there is provided a method of preparing a compound of method of preparing a compound of a compound of Formula 1 by reacting a compound of Formula 2

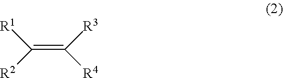

with an oxidising agent.

Preferred examples of oxidising agent include air, oxygen and oxygen containing compounds such as peroxides, per-salts and compounds of oxygen with other elements such as hypohalites. Preferably the oxidising agent comprises a hypohalite such as chlorite with an alcohol ROH; under basic reaction conditions at elevated temperature and pressure.

In formula 2 each R¹ to R⁴ is selected from the group consisting of F, Cl, H, CF₃, and C₁ to C₆ alkyl which may be at least partially fluorinated, wherein at least one of R¹ to R⁴ is or comprises F.

According to a tenth aspect of the invention there is provided a method of preparing a battery electrolyte formulation comprising mixing comprising a compound of Formula 1 with a lithium containing compound.

According to an eleventh aspect of the invention there is provided a method of improving battery capacity/charge transfer within a battery/battery life/etc by the use of a compound of Formula 1.

### Compound of Formula 1

In reference to all aspects of the invention the preferred embodiment of Formula (1) is below

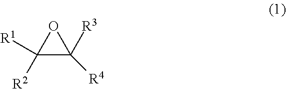

wherein each R¹ to R⁴ is selected from the group consisting of F, Cl, H, CF₃, and C₁ to C₆ alkyl which may be at least partially fluorinated, wherein at least one of R¹ to R⁴ is or comprises F.

### Advantages

In the aspects of the invention the electrolyte formulation has been found to be surprisingly advantageous.

The advantages of using compounds of Formula 1 in electrolyte solvent compositions manifest themselves in a number of ways. Their presence can reduce the flammability of the electrolyte composition (such as when for example measured by flashpoint). Their oxidative stability makes them useful for batteries required to work in harsh conditions and they are compatible with common electrode chemistries and can even enhance the performance of these electrodes through their interactions with them.

Additionally, electrolyte compositions comprising compounds of formula 1 have been found to have superior physical properties including low viscosity and a low melting point, yet a high boiling point with the associated advantage of little or no gas generation in use. The electrolyte formulation has been found to wet and spread extremely well over surfaces particularly fluorine containing surfaces; this is postulated to result from a beneficial a relationship between its adhesive and cohesive forces, to yield a low contact angle.

Furthermore, electrolyte compositions that comprise compounds of Formula 1 have been found to have superior electro-chemical properties including improved capacity retention, improved cyclability and capacity, improved compatibility with other battery components e.g. separators and current collectors and with all types of cathode and anode chemistries including systems that operate across a range of voltages and especially high voltages and which include additives such as silicon. In addition, the electrolyte formulations display good solvation of metal (e.g. lithium) salts and interaction with any other electrolyte solvents present.

Preferred features relating to the aspects of the invention follows below.

### Preferred Compounds

Preferred examples of compounds of the first embodiment of Formula 1

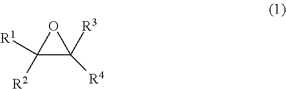

are where:
R¹ is H,
R² is CF₃,
R³ is F or CF₃, and
R⁴ is F or CF₃.

### Electrolyte Formulation

Preferably the electrolyte formulation comprises 0.1 wt % to 99.9 wt % of a compound of Formula 1. Optionally the compound of Formula 1 is present (in the electrolyte formulation) in an amount of more than 1 wt %, optionally more than 5 wt %, optionally more than 10 wt %, optionally more than 15 wt %, optionally more than 20 wt % and optionally more than 25 wt %. Optionally the compound of Formula 1 is present (in the electrolyte formulation) in an amount of less than 1 wt %, optionally less than 5 wt %, optionally less than 10 wt %, optionally less than 15 wt %, optionally less than 20 wt % and optionally less than 25 wt %.

### Metal Salts

The nonaqueous electrolytic solution further comprises a metal electrolyte salt, typically present in an amount of 0.1 to 20 wt % relative to the total mass of the nonaqueous electrolyte formulation.

The metal salt is preferably a salt of lithium, sodium, magnesium, calcium, lead, zinc or nickel.

Preferably the metal salt comprises a salt of lithium, such as those selected from the group comprising lithium hexafluorophosphate (LiPF₆), lithium perchlorate (LiClO₄), lithium tetrafluoroborate (LiBF₄), lithium triflate (LiSO₃CF₃), lithium bis(fluorosulfonyl)imide (Li(FSO₂)₂N) and lithium bis(trifluoromethanesulfonyl)imide (Li(CF₃SO₂)₂N).

### Solvents

The nonaqueous electrolytic solution may comprise a solvent. Preferred examples of solvents include fluoroethylene carbonate (FEC) and/or propylene carbonate (PC), dimethyl carbonate (DMC), ethylmethyl carbonate (EMC) or ethylene carbonate (EC).

Where present the solvent makes up from 0.1 wt % to 99.9 wt % of the liquid component of the electrolyte.

### Additives

The nonaqueous electrolytic solution may include an additive.

Suitable additives may serve as surface film-forming agents, which form an ion permeable film on the surface of the positive electrode or the negative electrode. This can pre-empt a decomposition reaction of the nonaqueous solvent and the electrolyte salt occurs on the surface of the electrodes, thereby preventing the decomposition reaction of the nonaqueous electrolytic solution on the surface of the electrodes.

Examples of film-forming agent additives include vinylene carbonate (VC), ethylene sulfite (ES), lithium bis(oxalato)borate (LiBOB), cyclohexylbenzene (CHB) and ortho-terphenyl (OTP). The additives may be used singly, or two or more may be used in combination.

When present the additive is present in an amount of 0.1 to 3 wt % relative to the total mass of the nonaqueous electrolyte formulation.

### Battery

### Primary/Secondary Battery

The battery may comprise a primary (non-rechargeable) or a secondary battery (rechargeable). Most preferably the battery comprises a secondary battery.

A battery comprising the nonaqueous electrolytic solutions will generally comprise several elements. Elements making up the preferred nonaqueous electrolyte secondary battery are described below. It is appreciated that other battery elements may be present (such as a temperature sensor), the list of battery components below is not intended to be exhaustive.

### Electrodes

The battery generally comprises a positive and negative electrode. Usually the electrodes are porous and permit metal ion (lithium ions) to move in and out of their structures with a process called insertion (intercalation) or extraction (deintercalation).

For rechargeable batteries (secondary batteries), the term cathode designates the electrode where reduction is taking place during the discharge cycle. For lithium-ion cells the positive electrode (“cathode”) is the lithium-based one.

### Positive Electrode (Cathode)

The positive electrode is generally composed of a positive electrode current collector such as a metal foil, optionally with a positive electrode active material layer disposed on the positive electrode current collector.

The positive electrode current collector may be a foil of a metal that is stable at a range of potentials applied to the positive electrode, or a film having a skin layer of a metal that is stable at a range of potentials applied to the positive electrode. Aluminium (Al) is desirable as the metal that is stable at a range of potentials applied to the positive electrode.

The positive electrode active material layer generally includes a positive electrode active material and other components such as a conductive agent and a binder. This is generally obtained by mixing the components in a solvent, applying the mixture onto the positive electrode current collector, followed by drying and rolling.

The positive electrode active material may be a lithium (Li) containing transition metal oxide. The transition metal element may be at least one selected from the group consisting of scandium (Sc), manganese (Mn), iron (Fe), cobalt (Co), nickel (Ni), copper (Cu) and yttrium (Y). Of these transition metal elements, manganese, cobalt and nickel are the most preferred.

Some of the transition metal atoms in the transition metal oxide may be replaced by atoms of a non-transition metal element. The non-transition element may be selected from the group consisting of magnesium (Mg), aluminium (Al), lead (Pb), antimony (Sb) and boron (B). Of these non-transition metal elements, magnesium and aluminium are the most preferred.

Preferred examples of positive electrode active materials include lithium-containing transition metal oxides such as LiCoO₂, LiNiO₂, LiMn₂O₄, LiMnO₂, LiNi_{1-y}Co_{y}O₂ (0<y<1), LiNi_{1-y-z}Co_{y}Mn_{z}O₂ (0<y+z<1) and LiNi_{1-y-z}Co_{y}A_{z}O₂ (0<y+z<1). LiNi1−y−zCo_{y}Mn_{z}O₂ (0<y+z<0.5) and LiNi_{1-y-z}Co_{y}Al_{z}O₂ (0<y+z<0.5) containing nickel in a proportion of not less than 50 mol % relative to all the transition metals are desirable from the perspective of cost and specific capacity. These positive electrode active materials contain a large amount of alkali components and thus accelerate the decomposition of nonaqueous electrolytic solutions to cause a decrease in durability. However, the nonaqueous electrolytic solution of the present disclosure is resistant to decomposition even when used in combination with these positive electrode active materials.

The positive electrode active material may be a lithium (Li) containing transition metal fluoride. The transition metal element may be at least one selected from the group consisting of scandium (Sc), manganese (Mn), iron (Fe), cobalt (Co), nickel (Ni), copper (Cu) and yttrium (Y). Of these transition metal elements, manganese, cobalt and nickel are the most preferred.

A conductive agent may be used to increase the electron conductivity of the positive electrode active material layer. Preferred examples of the conductive agents include conductive carbon materials, metal powders and organic materials. Specific examples include carbon materials as acetylene black, ketjen black and graphite, metal powders as aluminium powder, and organic materials as phenylene derivatives.

A binder may be used to ensure good contact between the positive electrode active material and the conductive agent, to increase the adhesion of the components such as the positive electrode active material with respect to the surface of the positive electrode current collector. Preferred examples of the binders include fluoropolymers and rubber polymers, such as polytetrafluoroethylene (PTFE), polyvinylidene fluoride (PVdF) ethylene-propylene-isoprene copolymer and ethylene-propylene-butadiene copolymer. The binder may be used in combination with a thickener such as carboxymethylcellulose (CMC) or polyethylene oxide (PEO).

### Negative Electrode (Anode)

The negative electrode is generally composed of a negative electrode current collector such as a metal foil, optionally with a negative electrode active material layer disposed on the negative electrode current collector.

The negative electrode current collector may be a foil of a metal. Copper (lithium free) is suitable as the metal. Copper is easily processed at low cost and has good electron conductivity.

Generally, the negative electrode comprises carbon, such as graphite or graphene.

Silicon based materials can also be used for the negative electrode. A preferred form of silicon is in the form of nano-wires, which are preferably present on a support material. The support material may comprise a metal (such as steel) or a non-metal such as carbon.

The negative electrode may include an active material layer. Where present the active material layer includes a negative electrode active material and other components such as a binder. This is generally obtained by mixing the components in a solvent, applying the mixture onto the positive electrode current collector, followed by drying and rolling.

Negative electrode active materials are not particularly limited, provided the materials can store and release lithium ions. Examples of suitable negative electrode active materials include carbon materials, metals, alloys, metal oxides, metal nitrides, and lithium-intercalated carbon and silicon. Examples of carbon materials include natural/artificial graphite, and pitch-based carbon fibres. Preferred examples of metals include lithium (Li), silicon (Si), tin (Sn), germanium (Ge), indium (In), gallium (Ga), lithium alloys, silicon alloys and tin alloys.

As with the positive electrode, the binder may be a fluoropolymer or a rubber polymer and is desirably a rubbery polymer, such as styrene-butadiene copolymer (SBR). The binder may be used in combination with a thickener.

### Separator

A separator is preferably present between the positive electrode and the negative electrode. The separator has insulating properties. The separator may comprise a porous film having ion permeability. Examples of porous films include microporous thin films, woven fabrics and nonwoven fabrics. Suitable materials for the separators are polyolefins, such as polyethylene and polypropylene.

### Case

The battery components are preferably disposed within a protective case.

The case may comprise any suitable material which is resilient to provide support to the battery and an electrical contact to the device being powered.

In one embodiment the case comprises a metal material, preferably in sheet form, moulded into a battery shape. The metal material preferably comprises a number of portions adaptable be fitted together (e.g. by push-fitting) in the assembly of the battery. Preferably the case comprises an iron/steel-based material.

In another embodiment the case comprises a plastics material, moulded into a battery shape. The plastics material preferably comprises a number of portions adaptable be joined together (e.g. by push-fitting/adhesion) in the assembly of the battery. Preferably the case comprises a polymer such as polystyrene, polyethylene, polyvinyl chloride, polyvinylidene chloride, or polymonochlorofluoroethylene. The case may also comprise other additives for the plastics material, such as fillers or plasticisers. In this embodiment wherein the case for the battery predominantly comprises a plastics material a portion of the casing may additionally comprise a conductive/metallic material to establish electrical contact with the device being powered by the battery.

### Arrangement

The positive electrode and negative electrode may be wound or stacked together through a separator. Together with the nonaqueous electrolytic solution they are accommodated in the exterior case. The positive and negative electrodes are electrically connected to the exterior case in separate portions thereof.

### Module/Pack

A number/plurality of battery cells may be made up into a battery module. In a battery module the battery cells may be organised in series and/or parallel. Typically these are encased in a mechanical structure.

A battery pack may be assembled by connecting multiple modules together in series or parallel. Typically battery packs include further features such as sensors and controllers including battery management systems and thermal management systems. The battery pack generally includes an encasing housing structure to make up the final battery pack product.

### End Uses

The battery of the invention, in the form an individual battery/cell, module and/or pack (and the electrolyte formulations therefor) are intended to be used in one or more of a variety of end products.

Preferred examples of end products include portable electronic devices, such as GPS navigation devices, cameras, laptops, tablets and mobile phones. Other preferred examples of end products include vehicular devices (as provision of power for the propulsion system and/or for any electrical system or devices present therein) such as electrical bicycles and motorbikes as well as automotive applications (including hybrid and purely electric vehicles).

The invention will now be illustrated with reference to the following non-limiting examples.

### EXAMPLE 1

### Typical Procedure for Epoxidation of Fluoroalkenes

A one litre round bottomed flask was equipped with a chilled condenser, magnetic stirrer bar, thermometer and dry ice trap.

The flask was charged with NaOCl (500 mL, 6-14% active Cl), Aliquat 336 (5 mL, 0.1 mol) and Xylenes (150 mL, 1.23 mol). This mixture was stirred at 600 rpm and allowed to cool to around 5° C. at which point Z-1,3,3,3-Tetrafluoropropene (50 g, 0.44 mol) was added dropwise over the course of 20 minutes. The reaction mixture was stirred for twenty-four hours whilst gradually warming to room temperature. After twenty-four hours the mixture was transferred to a separating funnel and allowed to separate. The aqueous layer was discarded, and the organic layer was dried over anhydrous sodium sulphate and filtered to remove the spent desiccant.

The product was recovered from the xylene solvent by distillation.

Several batches of material were prepared. Each was first concentrated by performing a crude single stage distillation prior to combining them for further purification by fractional distillation using a vacuum jacketed distillation column (50 cm*2 cm) equipped with a reflux divider and packed with Pro-pak 0.16 square inch 316 stainless steel distillation packing.

The reboiler was charged with a mixture comprising crude Z-1,3,3,3-tetrafluoropropene epoxide in xylene (251 g). The mixture was brought to reflux and the system allowed to equilibrate before the product was collected in 9 fractions. Each fraction was analysed by GC-MS. Fractions 1-4 and 9 were combined to give 60.8 g of a product comprising 81.8% of Z-1,3,3,3-tetrafluoropropene epoxide. Fractions 5-8 were combined to give 63.7 g of a product comprising 98.7% of Z-1,3,3,3-tetrafluoropropene epoxide:

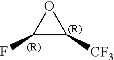

Z-1,3,3,3-tetrafluoropropene epoxide ((2R,3R)-2-Fluoro-3-(trifludiromethyl)oxirane): Boiling point 54-55° C.; MS m/z 130, 111, 82, 80, 69, 63, 60, 51, 47, 45, 33; ¹⁹F NMR (56 MHz) δ −70.73 (ddd, J 13.0, 5.0, 2.0 Hz, 3F), −165.27 to −168.36 (m, 1F).

### Flammability and Safety Testing

### Flash Point

Flashpoints were determined using a Miniflash FLP/H device from Grabner Instruments following the ASTM D6450 standard method:

| | Concentration (% wt) in standard electrolyte 1 M | | | | | |
|---|---|---|---|---|---|---|
| | LiPF₆ in (30% Ethylene carbonate & | | | | | |
| | 70% ethyl methyl carbonate) | | | | | |
| | 0 | 2 | 5 | 10 | 30 | 100 |
| Component | Flashpoint (° C.) | | | | | |
| 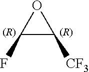 | 32 ± 2 | 32 ± 2 | 28 ± 1 | 30 ± 1 | 26 ± 2 | Not Detected |
| (MEXI-3) | | | | | | |

### Self-Extinguishing Time

Self-extinguishing time was measured with a custom-built device that contained an automatically controlled stopwatch connected to an ultraviolet light detector:
The electrolyte to be examined (500 μL) was applied to a Whatman GF/D (Ø=24 mm) glass microfiber filter
The ignition source was transferred under the sample and held in this its position for a preset time (1, 5 or 10 seconds) to ignite the sample. Ignition and burning of the sample were detected using a UV light detector.
Evaluation is carried out by plotting the burning time/weight of electrolyte [s g⁻¹] over ignition time [s] and extrapolation by linear regression line to ignition time=0 s
Self-extinguishing time (s·g⁻¹) is the time that is needed until the sample stops burning once inflamed

| | Concentration (% wt) in standard electrolyte 1 M LiPF₆ in | | | | | |
|---|---|---|---|---|---|---|
| | (30% Ethylene carbonate & 70% ethyl methyl carbonate) | | | | | |
| | 0 | 2 | 5 | 10 | 30 | 100 |
| Component | Self-extinguishing time (s.g⁻¹) | | | | | |
| 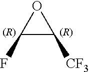 | 39 ± 2 | 25 ± 2 | 28 ± 2 | 28 ± 2 | 25 ± 2 | N.D.* |
| (MEXI-3) | | | | | | |

*compund took more than 10 seconds to ignite

These measurements demonstrate that the compound MEXI-3 has flame retarding properties.

### Electrochemical Testing

### Drying

Before testing MEXI-3 was dried by treatment with a pre-activated type 4A molecular sieve to less than 10 ppm water.

### Electrolyte Formulation

Electrolyte preparation and storage was carried out in an argon filled glove box (H₂O and O₂<0.1 ppm). The base electrolyte was 1M LiPF₆ in ethylene carbonate:ethyl methyl carbonate (30:70 wt. %) with MEXI-3 additive at concentrations of 2, 5, 10 and 30 wt. %.

### Cell Chemistry and Construction

The performance of each electrolyte formulation was tested in multi-layer pouch cells over 50 cycles (2 cells per electrolyte):

Chemistry 1: Lithium-Nickel-Cobalt-Manganese-Oxide (NCM622) positive electrode and artificial graphite (specific capacity: 350 mAh g⁻¹) negative electrode. The area capacity of NMC622 and graphite amounted to 3.5 mAh cm⁻² and 4.0 mAh cm⁻², respectively. The N/P ratio amounted to 115%.

Chemistry 2: Lithium-Nickel-Cobalt-Manganese-Oxide (NCM622) positive electrode and SiOₓ/graphite (specific capacity: 550 mAh g⁻¹) negative electrode. The area capacity of NMC622 and SiOₓ/graphite amount to 3.5 mAh/cm⁻² and 4.0 mAh cm⁻², respectively. The N/P ratio amounted to 115%

The test pouch cells had the following characteristics:
Nominal capacity 240 mAh+/−2%
Standard deviations:
Capacity: ±0.6 mAh
Coulombic Efficiency (CE) 1^{st} cycle: ±0.13%
Coulombic Efficiency (CE) subsequent cycles: ±0.1%
Positive electrode: NMC-622
   Active material content: 96.4%
   Mass loading: 16.7 mg cm⁻²
Negative electrode: Artificial Graphite
   Active material content: 94.8%
   Mass loading: 10 mg cm⁻²
   Separator: PE(16 μm)+4 μm Al₂O₃
   Balanced at cut-off voltage of 4.2 V
Negative electrode: Artificial graphite+SiO
   Active material content: 94.6%
   Mass loading: 6.28 mg cm⁻²
   Separator: PE(16 μm)+4 μm Al₂O₃
   Balanced at cut-off voltage of 4.2 V

After assembly the following formation protocol was used:
1. Step charge to 1.5 V followed by 5 h rest step (wetting step @ 40° C.)
2. CCCV (C/10, 3.7 V (Iₗᵢₘᵢₜ: 1 h)) (preformation step)
3. Rest step (6 h)
4. CCCV (C/10, 4.2 V (Iₗᵢₘᵢₜ: 0.05 C)) rest step (20 min)
5. CC discharge (C/10, 3.8 V), (degassing of the cell)
6. CC discharge (C/10, 2.8 V)

Following this formation step, the cells were tested as follows:
Rest step (1.5 V, 5 h), CCCV (C/10, 3.7 V (1 h))
Rest step (6 h), CCCV (C/10, 4.2 V (Iₗᵢₘᵢₜ: 0.05 C))
Rest step (20 min), CC discharge (C/10, 3.8 V)
Degassing step
Discharge (C/10, 2.8 V), rest step (5 h)
CCCV (C/3, 4.2 V (Iₗᵢₘᵢₜ: 0.05 C)), rest step (20 min)
CC discharge (C/3, 2.8 V)
50 cycles or until 50% SOH is reached at 40° C.:
CCCV (C/3, 4.2 V (Iₗᵢₘᵢₜ: 0.02 C)), rest step (20 min)
CC discharge (C/3, 3.0 V), rest step (20 min)

### Test Results

TABLE 1
Electrochemical performance of MEXI-3 - Cell Chemistry 1

| | | | Base electrolyte + | | Base electrolyte + | | Base electrolyte + | | Base electrolyte + | |
|---|---|---|---|---|---|---|---|---|---|---|
| | Base electrolyte | | 2 wt. % MEXI-3 | | 5 wt. % MEXI-3 | | 10 wt. % MEXI-3 | | 30 wt. % MEXI-3 | |
| | Discharge | Coulombic | Discharge | Coulombic | Discharge | Coulombic | Discharge | Coulombic | Discharge | Coulombic |
| Cycle | capacity | efficiency | capacity | efficiency | capacity | efficiency | capacity | efficiency | capacity | efficiency |
| No. | (mAh) | (%) | (mAh) | (%) | (mAh) | (%) | (mAh) | (%) | (mAh) | (%) |
| 1^{st} | 232.2 | 90.61 | 231.7 | 90.00 | 235.4 | 89.83 | 235.6 | 89.59 | 239.0 | 90.85 |
| (0.1 C) | | | | | | | | | | |
| 3^{rd} | 224.4 | 99.55 | 225.6 | 99.56 | 229.4 | 99.56 | 231.2 | 99.57 | 232.3 | 99.63 |
| (0.3 C) | | | | | | | | | | |
| 50^{th} | 218.1 | 99.83 | 219.5 | 99.82 | 223.1 | 99.81 | 225.4 | 99.89 | 226.9 | 99.95 |
| (0.3 C) | | | | | | | | | | |

TABLE 2
Electrochemical performance of MEXI-3 - Cell Chemistry 2

| | | | Base electrolyte + | | Base electrolyte + | | Base electrolyte + | | Base electrolyte + | |
|---|---|---|---|---|---|---|---|---|---|---|
| | Base electrolyte | | 2 wt. % MEXI-3 | | 5 wt. % MEXI-3 | | 10 wt. % MEXI-3 | | 30 wt. % MEXI-3 | |
| | Discharge | Coulombic | Discharge | Coulombic | Discharge | Coulombic | Discharge | Coulombic | Discharge | Coulombic |
| Cycle | capacity | efficiency | capacity | efficiency | capacity | efficiency | capacity | efficiency | capacity | efficiency |
| No. | (mAh) | (%) | (mAh) | (%) | (mAh) | (%) | (mAh) | (%) | (mAh) | (%) |
| 1^{st} | 199.6 | 74.52 | 199.0 | 74.25 | 197.8 | 73.82 | 198.1 | 74.37 | 199.7 | 74.68 |
| (0.1 C) | | | | | | | | | | |
| 3^{rd} | 176.3 | 97.03 | 176.9 | 97.16 | 176.9 | 97.19 | 177.1 | 97.40 | 178.9 | 97.54 |
| (0.3 C) | | | | | | | | | | |
| 50^{th} | 125.7 | 99.62 | 128.5 | 99.65 | 130.5 | 99.67 | 133.2 | 99.70 | 136.5 | 99.74 |
| (0.3 C) | | | | | | | | | | |

The test results for the additive MEXI-3 in each cell chemistry are summarised in Tables 1-2 and FIGS. 1-2. From this data it can be seen that the additive in both cell chemistries had a positive influence on cell performance improving both Coulombic efficiency and cycling stability. These results combined with the safety related studies demonstrate that the compounds of this invention simultaneously improved both the safety and performance of energy storage devices containing them.

### FIGURES

FIGS. 1-2 show the test results for the additive ETFMP in each cell chemistry.

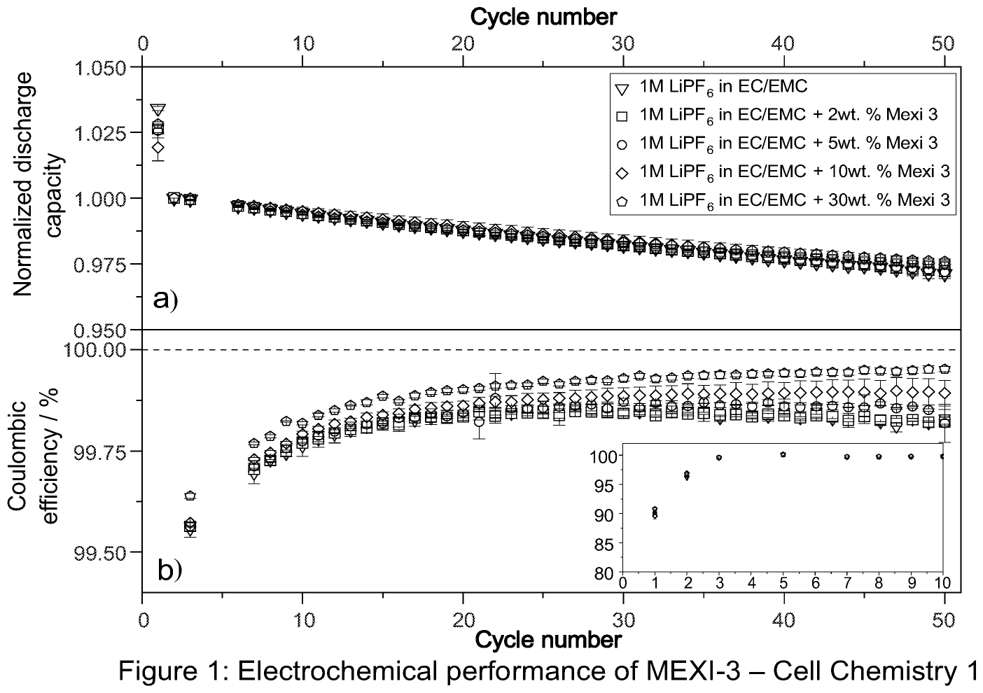

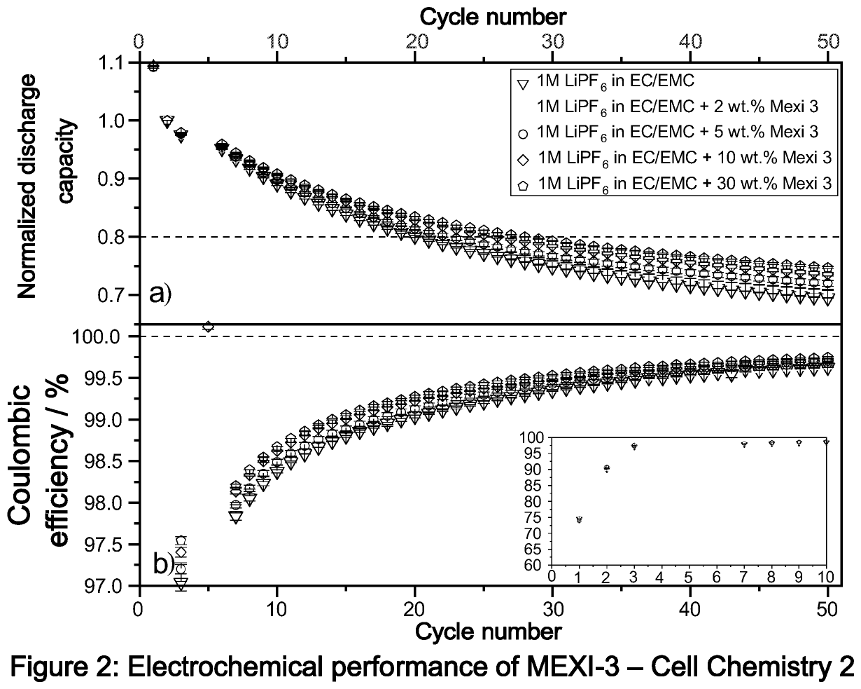

1. A nonaqueous battery electrolyte formulation, comprising a compound of Formula 1: 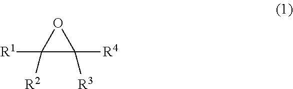
wherein each of R¹ to R⁴ is independently selected from the group consisting of F, Cl, H, CF₃, and C₁ to C₆ alkyl which is optionally at least partially fluorinated; and
wherein at least one of R¹ to R⁴ is or comprises F.

2. A battery, comprising the formulation according to claim 1.

3. The formulation according to claim 1, further comprising a metal electrolyte salt, present in an amount of from 0.1 to 20 wt % relative to a total mass of the formulation, wherein the metal salt is a salt of lithium, sodium, magnesium, calcium, lead, zinc, or nickel.

4. (canceled)

5. The formulation according to claim 3, wherein the metal salt is a salt of lithium selected from the group consisting of lithium hexafluorophosphate (LiPF₆), lithium hexafluoroarsenate monohydrate (LiAsF₆.H₂O), lithium perchlorate (LiClO₄), lithium tetrafluoroborate (LiBF₄), lithium triflate (LiSO₃CF₃), lithium bis(fluorosulfonyl)imide (Li(FSO₂)₂N), and lithium bis(trifluoromethanesulfonyl)imide (Li(CF₃SO₂)₂N).

6. The formulation according to claim 1, further comprising an additional solvent in an amount of from 0.1 wt % to 99.9 wt % of a liquid component of the formulation, wherein the additional solvent is selected from the group consisting of fluoroethylene carbonate (FEC), propylene carbonate (PC), ethylene carbonate (EC), and ethyl methyl carbonate (EMC).

7-8. (canceled)

9. The formulation according to claim 1, further comprising a metal ion, optionally in combination with a solvent.

10-15. (canceled)

16. A method of reducing the flammability of a battery and/or a battery electrolyte, the method comprising adding to the battery and/or the battery electrolyte the formulation according to claim 1.

17. A method of powering an article comprising a battery, the method comprising adding to the battery a battery electrolyte formulation comprising a compound of Formula 1: 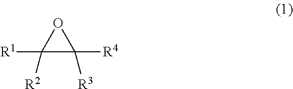
wherein each of R¹ to R⁴ is independently selected from the group consisting of F, Cl, H, CF₃, and C₁ to C₆ alkyl which is optionally at least partially fluorinated; and
wherein at least one of R¹ to R⁴ is or comprises F.

18. A method of retrofitting a battery electrolyte, the method comprising (a) at least partially replacing the battery electrolyte with the formulation according to claim 1; and/or (b) supplementing the battery electrolyte with the formulation.

19. A method of preparing a formulation containing the compound of according to claim 1, the method comprising by reacting a compound of Formula 2 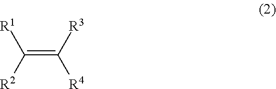
with an oxidizing agent so as to produce the compound.

20. A method of preparing the formulation according to claim 5, the method comprising mixing the compound of Formula 1 with dimethyl carbonate (DMC), fluoroethylene carbonate (FEC), propylene carbonate (PC), and ethylene carbonate (EC), or methyl ethyl carbonate (EMC) and with the salt of lithium so as to produce the formulation.

21. The method according to claim 17, wherein a capacity of the battery and/or a charge transfer within the battery is improved relative to a battery without the formulation.

22. The method according to claim 17, wherein the formulation further comprises a metal electrolyte salt, present in an amount of from 0.1 to 20 wt % relative to a total mass of the formulation, wherein the metal salt is a salt of lithium, sodium, magnesium, calcium, lead, zinc, or nickel.

23. (canceled)

24. The method according to claim 22, wherein the metal salt is a salt of lithium selected from the group consisting of lithium hexafluorophosphate (LiPF₆), lithium hexafluoroarsenate monohydrate (LiAsF₆.H₂O), lithium perchlorate (LiClO₄), lithium tetrafluoroborate (LiBF₄), lithium triflate (LiSO₃CF₃), lithium bis(fluorosulfonyl)imide (Li(FSO₂)₂N), and lithium bis(trifluoromethanesulfonyl)imide (Li(CF₃SP₂)₂N).

25. The method according to claim 17, wherein the formulation further comprises an additional solvent in an amount of from 0.1 wt to 99.9 wt % of a liquid component of the formulation, wherein the additional solvent is selected from the group consisting of dimethyl carbonate (DMC), fluoroethylene carbonate (FEC), propylene carbonate (PC), ethylene carbonate (EC), and methyl ethyl carbonate (EMC).

26. (canceled)

27. A nonaqueous battery electrolyte formulation, comprising a compound of Formula 1: 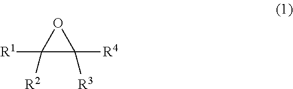
wherein R¹ is H;
R² is CF₃; and
each of R³ and R⁴ is independently F or CF₃.

28. A battery, comprising the formulation according to claim 27.

29. The formulation according to claim 27, further comprising a metal electrolyte salt, present in an amount of from 0.1 to 20 wt % relative to a total mass of the formulation, wherein the metal salt is a salt of lithium, sodium, magnesium, calcium, lead, zinc, or nickel.

30. The formulation according to claim 29, wherein the metal salt is a salt of lithium selected from the group consisting of lithium hexafluorophosphate (LiPF₆), lithium hexafluoroarsenate monohydrate (LiAsF₆.H₂O), lithium perchlorate (LiClO₄), lithium tetrafluoroborate (LiBF₄), lithium triflate (LiSO₃CF₃), lithium bis(fluorosulfonyl)imide (Li(FSO₂)₂N), and lithium bis(trifluoromethanesulfonyl)imide (Li(CF₃SO₂)₂N).

31. The formulation according to claim 27, further comprising an additional solvent in an amount of from 0.1 wt % to 99.9 wt % of a liquid component of the formulation, wherein the additional solvent is selected from the group consisting of fluoroethylene carbonate (FEC), propylene carbonate (PC), ethylene carbonate (EC), and methyl ethyl carbonate (EMC).
